(19) United States
(12) Patent Application Publication
(10) Pub. No.: US 2025/0099027 A1
(43) Pub. Date: Mar. 27, 2025

(54) SLEEP MANAGEMENT SYSTEM AND SLEEP MANAGEMENT METHOD

(71) Applicant: SAMSUNG ELECTRONICS CO., LTD., Suwon-si (KR)

(72) Inventors: Doyoon KIM, Suwon-si (KR); Chanwon LEE, Suwon-si (KR)

(73) Assignee: SAMSUNG ELECTRONICS CO., LTD., Suwon-si (KR)

(21) Appl. No.: 18/784,521

(22) Filed: Jul. 25, 2024

Related U.S. Application Data
(63) Continuation of application No. PCT/KR2024/010352, filed on Jul. 18, 2024.

(30) Foreign Application Priority Data
Sep. 27, 2023 (KR) 10-2023-0131107

(51) Int. Cl. A61B 5/00; A61B 5/0205; A61B 5/024; A61B 5/08; A61B 5/11

(52) U.S. Cl. CPC A61B 5/4812; A61B 5/0002; A61B 5/0205; A61B 5/1116; A61B 5/681; A61B 5/6891; A61B 5/7267; A61B 5/7282; A61B 5/743; A61B 5/746; A61B 5/02416; A61B 5/0816; A61B 5/14551; A61B 5/28; A61B 2560/045; A61B 2560/0462; A61B 2562/0219; A61B 2562/0247

(57) ABSTRACT

A sleep management system including: a plurality of sensors including a first sensor and a second sensor, the first sensor and the second sensor being configured to collect data of a user; a hub device configured to: receive first data from the first sensor and second data from the second sensor, obtain first processed data based on processing of the first data, and obtain second processed data based on processing of the second data; and a user device configured to: receive the first processed data and the second processed data from the hub device, obtain sleep state information about a sleep stage and a body movement of the user based on the first processed data and the second processed data, determine whether a Rapid Eye Movement Sleep Behavior Disorder (RBD) of the user occurs based on the body movement of the user.

### CROSS REFERENCE TO RELATED APPLICATION(S)

This application is a continuation of International Application No. PCT/KR2024/010352, filed on Jul. 18, 2024, in the Korean Intellectual Property Receiving Office, which is based on and claims priority to Korean Patent Application No. 10-2023-0131107, filed on Sep. 27, 2023, in the Korean Intellectual Property Office, the disclosures of which are incorporated by reference herein in their entireties.

### BACKGROUND

### 1. Field

The disclosure relates to a sleep management system and sleep management method.

### 2. Description of Related Art

Rapid Eye Movement (REM) Sleep Behavior Disorder (RBD) is a type of parasomnia in which abnormal behavior occurs during sleep.

RBD occurs during REM sleep, and dreams of patients with RBD often involve unpleasant or aggressive actions. The aggressive behavior in such dreams may be carried out, causing physical harm to the patient or their partner during the dream.

Conventionally, polysomnography (PSG) may performed in a hospital to test for subjects with RBD.

However, PSG is expensive and uncomfortable because may require subjects with RBD to wear various contact sensors on their bodies and to sleep in a hospital.

### SUMMARY

Provided is a sleep management system and sleep management method that may more conveniently and effectively screen for Rapid Eye Movement Sleep Behavior Disorder (RBD) which involves unconscious behavior during REM sleep.

Technical objects that can be achieved by the disclosure are not limited to the above-mentioned objects. Additional aspects will be set forth in part in the description which follows and, in part, will be apparent from the description, or may be learned by practice of the presented embodiments.

According to an aspect of the disclosure, a sleep management system may include: a plurality of sensors including a first sensor and a second sensor, the first sensor and the second sensor being configured to collect data of a user; a hub device configured to: receive first data from the first sensor and second data from the second sensor, obtain first processed data based on processing of the first data, and obtain second processed data based on processing of the second data; and a user device configured to: receive the first processed data and the second processed data from the hub device, obtain sleep state information about a sleep stage and a body movement of the user based on the first processed data and the second processed data, determine whether a Rapid Eye Movement Sleep Behavior Disorder (RBD) of the user occurs based on the body movement of the user and the sleep stage of the user, and perform a first operation based on an occurrence of the RBD.

The plurality of sensors may include at least two among a pressure sensor, a Ultra-Wideband (UWB) sensor, a radar sensor, a photoplethysmography (PPG) sensor, an electrocardiogram sensor, and an acceleration sensor.

The hub device may be further configured to: obtain the first processed data by inputting the first data to a first machine learning model, and obtain the second processed data by inputting the second data to a second machine learning model.

The user device may be further configured to determine that the RBD occurs based on the sleep stage of the user being a Rapid Eye Movement (REM) sleep stage and the body movement of the user being greater than a preset movement.

The first sensor may be a contact sensor, and the second sensor is a non-contact sensor.

The user device may be further configured to: based on the sleep stage of the user being the REM sleep stage, obtain information about a first body movement of the user by inputting the first processed data, corresponding to the first data collected by the contact sensor, to a machine learning model, obtain information about a second body movement of the user by inputting the second processed data, corresponding to the second data collected by the non-contact sensor, to the machine learning model, and determine whether the RBD occurs based on the first body movement and the second body movement being greater than a preset movement.

The first operation may include an operation of alerting the user of the occurrence of the RBD via a user interface.

The first operation may include an operation of notifying a medical institution of the occurrence of the RBD.

The sleep management system may further include: a server device configured to receive RBD information from the user device.

The server device may be further configured to control at least one home appliance to perform a second operation to alleviate the RBD based on the occurrence of the RBD.

The server device may be further configured to control at least one home appliance to perform a third operation to wake up the user based on the occurrence of the RBD.

The user device may be further configured to: based on the occurrence of the RBD, perform at least one of an operation of alerting the user of the occurrence of the RBD, or an operation of transmitting the RBD information to a medical institution.

According to an aspect of the disclosure, a sleep management method may include: receiving, by a hub device, first data from a first sensor and second data from a second sensor; obtaining, by the hub device, first processed data based on processing of the first data, and second processed data based on processing of the second data; transmitting, by the hub device, the first processed data and the second processed data to a user device; obtaining, by the user device, sleep state information about a sleep stage and a body movement of a user based on the first processed data and the second processed data received from the hub device; determining, by the user device, whether a Rapid Eye Movement Sleep Behavior Disorder (RBD) of the user occurs based on the body movement of the user and the sleep stage of the user; and performing, by the user device, a first operation based on an occurrence of the RBD.

The determining whether the RBD of the user occurs may include: determining that the sleep stage of the user is the REM sleep stage and the body movement of the user is greater than a preset movement.

The first sensor may be a contact sensor, the second sensor may be a non-contact sensor, where the determining whether the RBD of the user occurs includes: based on the sleep stage of the user being the REM sleep stage, obtaining information about a first body movement of the user by inputting the first processed data, corresponding to the first data collected by the contact sensor, to a machine learning model, obtaining information about a second body movement of the user by inputting the second processed data, corresponding to the second data collected by the non-contact sensor, to the machine learning model, and determining that the RBD occurs based on the first body movement and the second body movement being greater than a preset movement.

According to an aspect of the disclosure, a non-transitory computer-readable medium having instructions stored therein, which when executed by one or more processors cause the one or more processors to execute a sleep management method, the sleep management method may include: receiving, by a hub device, first data from a first sensor and second data from a second sensor; obtaining, by the hub device, first processed data based on processing of the first data, and second processed data based on processing of the second data; transmitting, by the hub device, the first processed data and the second processed data to a user device; obtaining, by the user device, sleep state information about a sleep stage and a body movement of a user based on the first processed data and the second processed data received from the hub device; determining, by the user device, whether a Rapid Eye Movement Sleep Behavior Disorder (RBD) of the user occurs based the sleep state information; and performing at least one operation based on an occurrence of the RBD.

The determining whether the RBD occurs may include: determining that the sleep stage of the user is the REM sleep stage and the body movement of the user is greater than a preset movement.

The at least one operation may include one or more of: notifying a medical institution of the occurrence of the RBD, notifying the user of the occurrence of RBD, and control at least one home appliance to address the occurrence of the RBD.

### BRIEF DESCRIPTION OF DRAWINGS

The above and other aspects, features, and advantages of certain embodiments of the present disclosure will be more apparent from the following description taken in conjunction with the accompanying drawings, in which:

FIG. 1 illustrates an example of a network of a sleep management system according to an embodiment;

FIG. 2 schematically illustrates an example of a structure of a sleep management system according to an embodiment;

FIG. 3 illustrates an example of a control block diagram of a sleep management system according to an embodiment;

FIG. 4 and FIG. 5 illustrate an example of a plurality of sensors of a sleep management system according to an embodiment;

FIG. 6 is a flowchart illustrating an example of a sleep management method according to an embodiment;

FIG. 7 is a flowchart illustrating another example of a sleep management method according to an embodiment;

FIG. 8 is a diagram for illustrating a procedure for processing data collected from a plurality of sensors of a sleep management system according to an embodiment;

FIG. 9 is a diagram for illustrating sleep stages;

FIG. 10 illustrates an example of obtaining Rapid Eye Movement Sleep Behavior Disorder (RBD) information of a user in a sleep management method according to an embodiment;

FIG. 11 illustrates an example of sleep state information and RBD information derived by a sleep management system according to an embodiment;

FIG. 12 illustrates an example of preset operations following an occurrence of RBD of a user in a sleep management method according to an embodiment;

FIG. 13 illustrates an example of a preset operation to alleviate RBD of a user according to an embodiment;

FIG. 14 illustrates another example of a preset operation to alleviate RBD of a user according to an embodiment; and

FIG. 15 illustrates an example of a preset operation to induce wakeup of a user according to an embodiment.

### DETAILED DESCRIPTION

Hereinafter, example embodiments of the disclosure will be described in detail with reference to the accompanying drawings. The same reference numerals are used for the same components in the drawings, and redundant descriptions thereof will be omitted. The embodiments described herein are example embodiments, and thus, the disclosure is not limited thereto and may be realized in various other forms. It is to be understood that singular forms include plural referents unless the context clearly dictates otherwise. The terms including technical or scientific terms used in the disclosure may have the same meanings as generally understood by those skilled in the art.

It is understood that various embodiments of the disclosure and associated terms are not intended to limit technical features herein to particular embodiments, but encompass various changes, equivalents, or substitutions.

The singular form of a noun corresponding to an item may include one or more items unless the context states otherwise.

Throughout the specification, “A or B”, “at least one of A and B”, “at least one of A or B”, “A, B or C”, “at least one of A, B and C”, and “at least one of A, B, or C” may each include any one of, or all the possible combinations of A, B and C.

The expressions “and/or” and “or” are interpreted to include a combination or any of associated elements.

For example, the expression “A, B or C” may include one of A, B, and C or any combination thereof.

It will be understood that the terms “first”, “second”, or the like, may be used only to distinguish one component from another, not intended to limit the corresponding component in other aspects (e.g., importance or order).

When it is said that one (e.g., first) component is “coupled” or “connected” to another (e.g., second) component, with or without the terms “functionally” or “communicatively”, it means that one component may be connected to the other component directly (e.g., by wire), wirelessly, or through a third component.

It will be understood that the terms “has,” “having,” “includes,” “comprises,” “including,” and “comprising,” when used in this specification, specify the presence of stated features, figures, steps, operations, components, members, or combinations thereof, but do not preclude the presence or addition of one or more other features, figures, steps, operations, components, members, or combinations thereof.

An expression that one component is “connected”, “coupled”, “supported”, or “in contact” with another component includes a case in which the components are directly “connected”, “coupled”, “supported”, or “in contact” with each other and a case in which the components are indirectly “connected”, “coupled”, “supported”, or “in contact” with each other through a third component.

It will also be understood that when one component is referred to as being “on” or “over” another component, it may be directly on the other component or intervening components may also be present.

Reference numerals used for method steps are simply used for convenience of description, but not to limit an order of the steps. Thus, unless the context clearly dictates otherwise, the written order may be practiced otherwise.

Hereinafter, a sleep management system according to various embodiments will be described in detail with reference to the accompanying drawings.

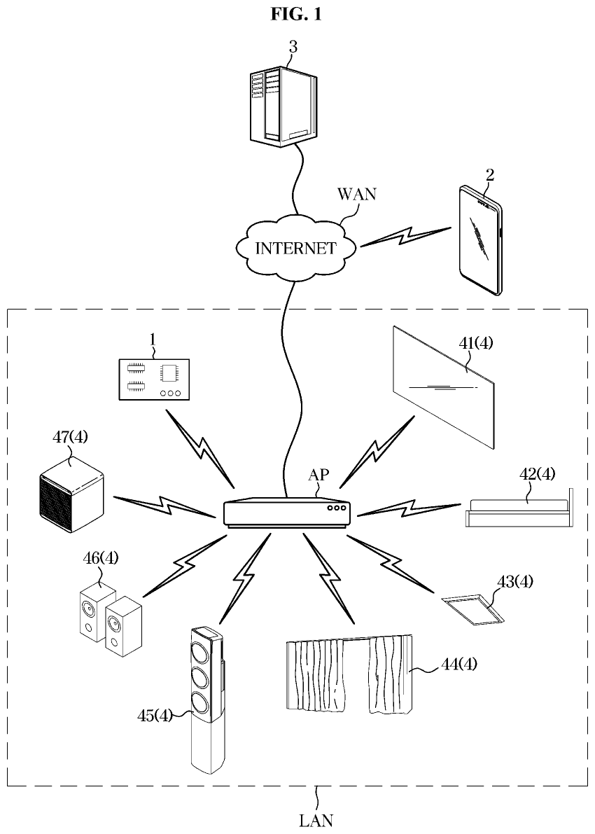

FIG. 1 illustrates an example of a network of a sleep management system according to an embodiment.

Referring to FIG. 1, a sleep management system according to an embodiment may include a hub device 1 and a user device 2.

In addition, the sleep management system according to an embodiment may include a server device 3 and/or home appliances 4.

The hub device 1 may include a communication module capable of communicating with the user device 2, the server device 3 and/or the home appliances 4, at least one processor for processing data, and at least one memory that stores a program for controlling operation of the hub device 1.

The hub device 1 may obtain processed data based on processing of data collected from a plurality of sensors. According to an embodiment, the hub device 1 may use a machine learning model to process the data collected from the plurality of sensors.

According to an embodiment, the hub device 1 may transmit the processed data to the user device 2. For example, the hub device 1 may transmit the processed data to the user device 2 not through the server device 3, but by direct communication.

The home appliances 4 may include various types of electronic products. For example, the home appliances 4 may include at least one of a display device 41, a furniture control device 42, a lighting device 43, an automatic curtain open/close device 44, an air conditioner 45, a speaker 46 or an air purifier 47. The aforementioned home appliances are merely examples, and other various types of electronic products such as a clothes care apparatus in addition to the aforementioned home appliance products may also be included in the home appliances 4.

The home appliance 4 may be controlled remotely by the server device 3.

The furniture control device 42 may include an actuator that may change a posture of the user by changing the structure of the furniture, and/or a vibration element that may transmit vibration to the user who lies or sits on the furniture. For example, the furniture control device 42 may include an actuator that may control a reclining angle of a recliner bed, a recliner chair and/or a recliner sofa.

The lighting device 43 may include a light source with a controllable intensity and/or color of light.

The automatic curtain open/close device 44 may include an actuator for automatically opening or closing a curtain.

The server device 3 may include a communication module for communicating with the hub device 1, the user device 2 and/or the home appliance 4.

The server device 3 may include at least one processor that may process data received from the hub device 1, the user device 2 and/or the home appliances 4, and at least one memory that may store a program for processing data or processed data. The server device 3 may be implemented with various computing devices such as a workstation, a cloud, a data drive, a data station, etc. The server device 3 may be implemented with one or more servers physically or logically classified based on function, sub-configuration of the function or data, and may transmit or receive data through inter-server communication and process the data.

The server device 3 may perform functions of storing and/or managing a user account, registering the hub device 1, the user device 2 and/or the home appliance 4 by associating them with the user account, and managing or controlling the registered hub device 1 and the home appliance 4. For example, the user may access the server device 3 through the user device 2 to create a user account. The user account may be identified by an identity (ID) and a password created by the user. The user may access the server device 3 through the user device 2 to manage the user account. The server device 3 may register the hub device 1, the user device 2 and/or the home appliance 4 with the user account, according to a set procedure. For example, the server device 3 may connect identification information (e.g., a serial number, a media access control (MAC) address, etc.) of the hub device 1 to the user account to register, manage and control the hub device 1. Likewise, the server device 3 may register the user device 2 and the home appliance 4 with the user account and control them.

The server device 3 may receive various information from the hub device 1, the user device 2 and/or the home appliance 4 registered with the user account.

For example, the server device 3 may include a first server and a second server. The first server may create and/or manage user account information, and register and/or manage information about the hub device 1, the user device 2 and/or the home appliance 4 with the user account. The second server may receive registration information of the user device 2 and the home appliance 4 from the first server to control the user device 2 and/or the home appliance 4.

In another example, the second server may perform a function of managing the hub device 1 and the home appliance 4 registered in the first server on behalf of the first server.

The number of the server devices 3 is not limited thereto, and the server device 3 may include a plurality of servers for performing the same and/or different operations.

The user device 2 may include a communication module for communicating with the hub device 1, the server device 3 and/or the home appliance 4. The user device 2 may include a user interface for receiving user inputs or outputting information for the user. The user device 2 may include at least one processor for controlling operation of the user device 2 and at least one memory for storing a program for controlling the operation of the user device 2.

The user device 2 may be carried by the user or placed at the user's home or office. The user device 2 may include a personal computer, a terminal, a mobile phone, a smart phone, a handheld device, a wearable device, a display device, etc., without being limited thereto.

In the memory of the user device 2, a program, i.e., an application for processing data received from the hub device 1 may be stored. The application may be sold in a state of being installed in the user device 2, or may be downloaded and installed from an external server.

The user may access the server device 3 and create a user account by running the application installed in the user device 2, and register the hub device 1 and/or the home appliance 4 by communicating with the server device 3 based on the login user account.

For example, by operating the home appliance 4 to enable the home appliance 4 to access the server device 3 according to a procedure guided by the application installed in the user device 2, the server device 3 may register the home appliance 4 with the user account by assigning the identification information (e.g., a serial number or a MAC address) of the home appliance 4 with the user account. The home appliance 4 may also be registered with the user account in the similar manner. Information other than the serial number or MAC address of the device to identify the device may be used for the information required to register the device such as the home appliance 4 with the user account.

The user device 2 may receive various information from the hub device 1 and the home appliance 4 registered with the user account directly or through the server device 3.

A network may include both a wired network and a wireless network. The wired network may include a cable network or a telephone network, and the wireless network may include any network that transmits or receives signals in radio waves. The wired network and the wireless network may be connected to each other.

The network may include a Wide Area Network (WAN) such as the Internet, a Local Area Network (LAN) formed around an Access Point (AP), and a short-range wireless network without an AP. The short-range wireless network may include Bluetooth (IEEE 802.15.1), Zigbee (IEEE 802.15.4), wireless fidelity (Wi-Fi) direct, Near Field Communication (NFC), Z-wave, etc., without being limited thereto.

The AP may connect the hub device 1, the user device 2 and/or the home appliance 4 to the WAN connected to the server device 3. The hub device 1, the user device 2 and/or the home appliance 4 may be connected to the server device 3 through the WAN.

The AP may use wireless communication such as Wi-Fi (IEEE 802.11), Bluetooth (IEEE 802.15.1), Zigbee (IEEE 802.15.4), etc., to communicate with the hub device 1, the user device 2 and/or the home appliance 4, and use wired communication to access the WAN, but the wireless communication scheme of the AP is not limited thereto.

According to an embodiment, the hub device 1 may communicate with the user device 2 over a short-range wireless network without going through the AP.

For example, the hub device 1 may be connected to the user device 2 over a short-range wireless network (e.g., Wi-Fi direct, Bluetooth or NFC). In another example, the hub device 1 may use the long-range wireless network (e.g., a cellular communication module or the like) to be connected to the user device 2 through the WAN.

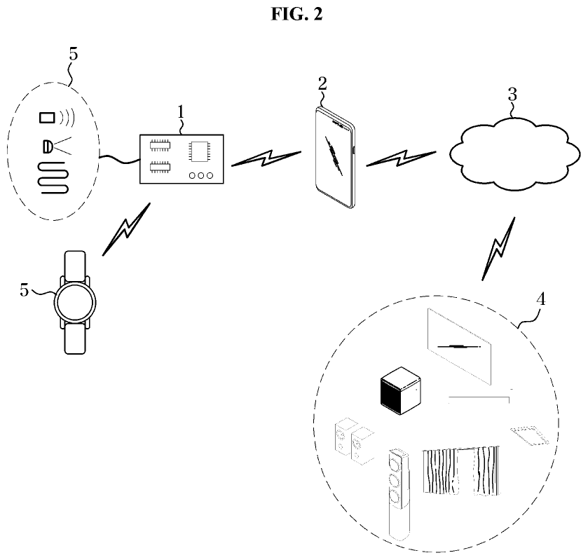

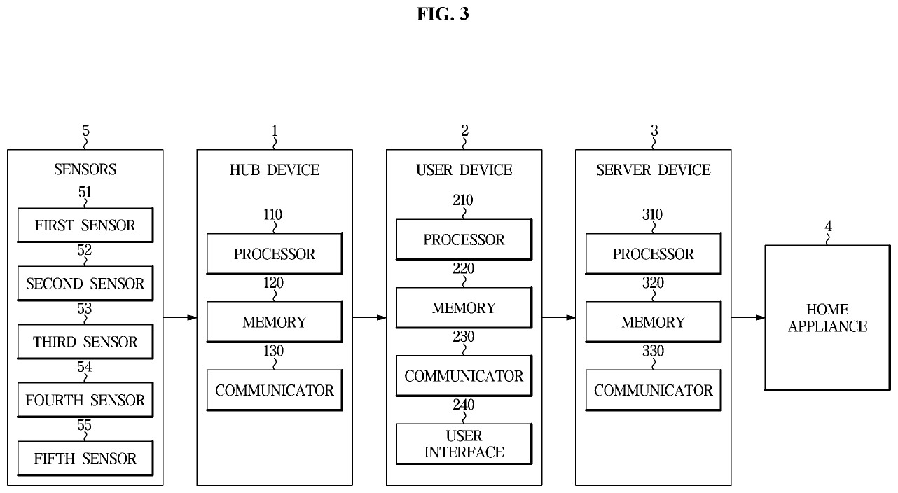

FIG. 2 schematically illustrates an example of a structure of a sleep management system according to an embodiment. FIG. 3 illustrates an example of a control block diagram of a sleep management system according to an embodiment.

Referring to FIG. 2 and FIG. 3, the hub device 1 may receive data collected from a plurality of sensors 5.

The plurality of sensors 5 may include sensors (e.g., a first sensor 51, a second sensor 52, a third sensor 53, a fourth sensor 54 and/or a fifth sensor 55) for collecting data of the user.

The plurality of sensors 5 may respectively collect data of the user and transmit the collected data of the user to the hub device 1.

According to an embodiment, the data of the user may include data associated with the user's sleep.

The data associated with the user's sleep may include pressure data for measuring a pressure change corresponding to a change in posture of the user, displacement data corresponding to displacement of the body that changes according to the user's breathing, heart rate data corresponding to the user's heart rate, oxygen saturation data corresponding to the user's oxygen saturation, electrocardiogram data corresponding to the user's electrocardiogram, acceleration data corresponding to acceleration that changes according to the user's movement, and/or eye-movement data corresponding to the movement of the user's eyes.

The plurality of sensors 5 may include at least two of a pressure sensor for collecting pressure data for measuring a pressure change corresponding to a change in the user's posture, a Ultra-Wideband (UWB) sensor for measuring displacement data corresponding to displacement of the body that changes according to the user's breathing, a photoplethysmography (PPG) sensor for collecting heart rate data corresponding to the user's heart rate and/or oxygen saturation data corresponding to the user's oxygen saturation, an electrocardiogram sensor for collecting electrocardiogram data corresponding to the user's electrocardiogram, an acceleration sensor for collecting acceleration data corresponding to acceleration that changes according to the user's movement, and/or a radar sensor for collecting eye-movement data corresponding to the user's eye movement.

The terms first, second, third, fourth, and fifth from the expressions, the first sensor 51, the second sensor 52, the third sensor 53, the fourth sensor 54, and the fifth sensor 55 indicate that the respective sensors are different sensors.

Each of the first sensor 51, the second sensor 52, the third sensor 53, the fourth sensor 54, and the fifth sensor 55 may be one of the pressure sensor, the UWB sensor, the radar sensor, the electrocardiogram sensor, the PPG sensor, or the acceleration sensor. The PPG sensor may include an oxygen saturation sensor and/or a heart rate sensor.

The first sensor 51, the second sensor 52, the third sensor 53, the fourth sensor 54, and the fifth sensor 55 may be divided into a contact sensor and a non-contact sensor.

For example, the pressure sensor, the PPG sensor, the electrocardiogram sensor, or the acceleration sensor may be a contact sensor. The UWB sensor or the radar sensor may be a non-contact sensor.

The plurality of sensors may include at least one contact sensor and at least one non-contact sensor.

Hereinafter, for convenience of description, the first sensor 51 is defined as the pressure sensor, the second sensor 52 is defined as the UWB sensor, the third sensor 53 is defined as the radar sensor, the fourth sensor 54 is defined as at least one of the PPG sensor and the electrocardiogram sensor, and the fifth sensor 55 is defined as the acceleration sensor.

The plurality of sensors 5 may further include an extra sensor (e.g., a microphone or camera) in addition to the first to fifth sensors 51 to 55, or may not include at least one of the first to fifth sensors 51 to 55.

Data collected from the plurality of sensors 5 may be transmitted to the hub device 1.

According to an embodiment, data collected by at least one of the plurality of sensors 5 may be transmitted to the hub device 1 by wired communication, and data collected by the other sensor(s) may be transmitted to the hub device 1 by wireless communication.

Accordingly, the hub device 1 may be wiredly connected to at least one of the plurality of sensors 5 and wirelessly connected to the other sensor(s).

According to an embodiment, the fourth sensor 54 may be included in a smart sensor device (e.g., a wearable device). The smart sensor device may include a wireless communication module and the fourth sensor 54. For example, the smart sensor device may include a smart watch that is shaped like a watch and/or a smart ring that is shaped like a ring, but the form of the smart sensor device is not limited thereto.

The smart sensor device may establish wireless communication with the hub device 1, and transmit data collected from the fourth sensor 54 to the hub device 1 by wireless communication.

In an embodiment, the smart sensor device may include the fourth sensor 54 and the fifth sensor 55. The smart sensor device may establish wireless communication with the hub device 1, and transmit data collected from the fourth sensor 54 and the fifth sensor 55 to the hub device 1 by wireless communication.

In an embodiment, the data collected from the plurality of sensors 5 may be transmitted to the hub device 1 by wired communication.

In various embodiments, the plurality of sensors 5 may include an imaging sensor (e.g., a camera). However, in an embodiment, the plurality of sensors 5 may all be non-imaging sensors.

In an embodiment of the disclosure, as all of the plurality of sensors 5 correspond to non-imaging sensors, an invasion of the user's privacy may be prevented.

In an embodiment, the hub device 1 may include at least one memory 120 for storing a program for processing data collected from the plurality of sensors 5, and at least one processor 110 that may process the data collected from the plurality of sensors 5 based on the program stored in the at least one memory 120.

The at least one memory 120 may store a machine learning model for processing the data collected from the plurality of sensors 5.

In an embodiment, the machine learning model may be a machine learning model for feature extraction, which extracts a feature of the data collected from the plurality of sensors 5 in response to input of the data, and outputs processed data including the extracted feature.

The feature of the data may include elements extracted from the data by the machine learning model to perform classification or prediction.

In an embodiment, the machine learning model may also be a machine learning model for sleep stage determination, which outputs processed data including data of a sleep stage of the user, in response to input of the data collected from the plurality of sensors 5.

For example, the at least one memory 120 may include a first machine learning model 11 for processing first data collected from the first sensor 51, a second machine learning model 12 for processing second data collected from the second sensor 52, a third machine learning model 13 for processing third data collected from the third sensor 53, a fourth machine learning model 14 for processing fourth data collected from the fourth sensor 54, and a fifth machine learning model 15 for processing fifth data collected from the fifth sensor 55.

The at least one processor 110 may obtain first processed data based on processing of the first data collected from the first sensor 51.

The first processed data may include feature data extracted from the first data and/or data about a sleep stage extracted from the first data. The volume of the first processed data may be smaller than the volume of the first data.

The at least one processor 110 may obtain second processed data based on processing of the second data collected from the second sensor 52.

The second processed data may include feature data extracted from the second data and/or data about a sleep stage extracted from the second data. The volume of the second processed data may be smaller than the volume of the second data.

The at least one processor 110 may obtain third processed data based on processing of the third data collected from the third sensor 53.

The third processed data may include feature data extracted from the third data and/or data about a sleep stage extracted from the third data. The volume of the third processed data may be smaller than the volume of the third data.

The at least one processor 110 may obtain fourth processed data based on processing of the fourth data collected from the fourth sensor 54.

The fourth processed data may include feature data extracted from the fourth data and/or data about a sleep stage extracted from the fourth data. The volume of the fourth processed data may be smaller than the volume of the fourth data.

The at least one processor 110 may obtain fifth processed data based on processing of the fifth data collected from the fifth sensor 55.

The fifth processed data may include feature data extracted from the fifth data and/or data about a sleep stage extracted from the fifth data. The volume of the fifth processed data may be smaller than the volume of the fifth data.

In the disclosure, the hub device 1 primarily processes data and then transmits it to the user device 2, thereby reducing data throughput to be borne by the user device 2.

The hub device 1 may include a communicator 130 including a wired communication module for performing wired communication with the plurality of sensors 5, and/or a wireless communication module for performing wireless communication with the user device 2, the server device 3 and/or the home appliances.

The hub device 1 may include a Printed Circuit Board (PCB) including the at least one processor 110, the at least one memory 120 and the communicator 130. At least some of the plurality of sensors 5 may be wiredly connected to the PCB.

The hub device 1 may include a housing that covers the PCB.

The hub device 1 may be installed in a location where user operation is difficult. Hence, in an embodiment, the hub device 1 may not include any user interface device (input/output device).

Based on a case in which the hub device 1 is installed in a place where the user may easily operate the hub device 1, in an embodiment, the hub device 1 may include a user interface device (input/output device).

In an embodiment, the user may operate the user interface device disposed in the hub device 1 to connect the hub device 1 to an Access Point (AP).

In an embodiment, the user may operate the user interface device disposed in the hub device 1 to activate the communicator 130 of the hub device 1.

In an embodiment, the user may operate the user interface device disposed in the hub device 1 to power on the hub device 1.

The at least one processor 110 may control the plurality of sensors 5.

For example, the at least one processor 110 may control at least one of the plurality of sensors 5 connected via wires.

The at least one processor 110 may wake up at least one of the plurality of sensors 5 connected via wires based on a sensor wakeup condition being satisfied.

The waking up of the sensor may include activating the sensor.

The at least one processor 110 may switch at least one of the plurality of sensors 5 connected via wires to a standby state based on a sensor standby condition being satisfied. The switching of the sensor to the standby state may include inactivating the sensor or driving the sensor in a low power mode.

The user device 2 may receive data obtained by the hub device 1 via wireless communication from the hub device 1.

The data obtained by the hub device 1 may include processed data resulting from processing of the data collected from the plurality of sensors 5.

In an embodiment, the user device 2 may include at least one memory 220 for storing a program for processing data received from the hub device 1, and at least one processor 210 that may process the data received from the hub device 1 based on the program stored in the at least one memory 220.

The at least one memory 220 may store a machine learning model for processing the data received from the hub device 1.

The at least one memory 220 may store a sleep management application that is downloadable from an external server. The sleep management application may be a downloadable app, at least a portion of which may be at least temporarily stored or arbitrarily created in a recording medium that may be readable to a device such as a server of the manufacturer, a server of the application store, or a relay server.

The sleep management application may include a machine learning model. The machine learning model included in the sleep management application may be updated by the external server.

In an embodiment, the user device 2 may include a communicator 230 including at least one communication module for establishing communication with the hub device 1, the server device 3, the home appliance 4 and/or the smart sensor device including at least some of the plurality of sensors 5.

The user device 2 may receive the processed data from the hub device 1 through the communicator 230.

In an embodiment, the at least one processor 210 may establish communication between the communicator 130 of the hub device 1 and the communicator 230 (e.g., a short-range wireless communication module) of the user device 2 in response to the communicator 230 being activated.

In an embodiment, the at least one processor 210 may control a user interface 240 to provide feedback that requests activation of the communicator 230 in response to the sleep management application being executed while the communicator 230 (e.g., the short-range wireless communication module) is not activated.

The at least one processor 210 may wirelessly receive data from the hub device 1 through the communicator 230 (e.g., the short-range wireless communication module).

The at least one processor 210 may use the machine learning model stored in the at least one memory 220 to process the data received from the hub device 1.

The data received from the hub device 1 may include the first processed data, the second processed data, the third processed data, the fourth processed data and/or the fifth processed data.

The at least one processor 210 may obtain sleep state information associated with a sleep state of the user based on the first processed data, the second processed data, the third processed data, the fourth processed data and/or the fifth processed data.

The at least one processor 210 may obtain the sleep state information associated with the user's sleep state by inputting the first processed data, the second processed data, the third processed data, the fourth processed data and/or the fifth processed data to the machine learning model stored in the at least one memory 220.

The sleep state information associated with the user's sleep state may include sleep state information associated with the user's sleep stage and the user's body movement.

The sleep state information associated with the user's sleep stage and the user's body movement may include information about the user's sleep stage and information about the user's body movement.

The machine learning model stored in the at least one memory 220 may be a machine learning model for determining a user's sleep state, which outputs sleep state information associated with the user's sleep state in response to input of the first processed data, the second processed data, the third processed data, the fourth processed data and/or the fifth processed data.

The at least one processor 210 may store, in the at least one memory 220, the sleep state information obtained by inputting the first processed data, the second processed data, the third processed data, the fourth processed data and/or the fifth processed data to the machine learning model.

The at least one processor 210 may determine whether Rapid Eye Movement (REM) Sleep Behavior Disorder (RBD) of the user occurs based on the user's sleep stage and the user's body movement.

RBD occurs during a REM sleep stage, and RBD may be accompanied by aggressive and violent behavior. As a result, a person with RBD may physically harm themselves, or hurt their partner while sleeping. RBD may be diagnosed if he or she exhibits actions that could be dangerous such as arm flailing, kicking, etc., or exhibits dream enactment behavior acting out his or her dreams during REM sleep.

The at least one processor 210 may obtain information about a first body movement of the user by inputting at least one processed data corresponding to data collected by the contact sensor from among the first processed data, the second processed data, the third processed data, the fourth processed data, and/or the fifth processed data to the machine learning model. In addition, the at least one processor 210 may obtain information about a second body movement of the user by inputting at least one processed data corresponding to data collected by the non-contact sensor from among the first processed data, the second processed data, the third processed data, the fourth processed data, and/or the fifth processed data to the machine learning model.

The information about the first body movement may include at least one of information about the presence or absence of the user's first body movement, information about an intensity of the first body movement, information about a size of the first body movement, or information about a motion of the first body movement.

The information about the second body movement may include at least one of information about the presence or absence of the second body movement, information about an intensity of the second body movement, information about a size of the second body movement, or information about a motion of the second body movement.

The at least one processor 210 may determine whether both the first body movement and the second body movement are greater than a preset movement based on the information about the user's first body movement and the information about the user's second body movement when the user's sleep stage is a REM sleep stage. The at least one processor 210 may determine that RBD occurs in response to both the first body movement and the second body movement being greater than the preset movement.

The at least one processor 210 may obtain RBD information associated with the user's RBD in response to the occurrence of the user's RBD.

The at least one processor 210 may store the RBD information of the user in the at least one memory 220.

The RBD information of the user may include at least one of information about whether RBD occurs, information about a degree of RBD, or information about an occurrence time of RBD.

The at least one processor 210 may control at least one of the communicator 230 or the user interface 240 to perform a preset operation in response to the occurrence of RBD.

In response to the occurrence of RBD, the at least one processor 210 may notify a medical institution registered in conjunction with the user device 2 of the occurrence of RBD via the communicator 230 (e.g., a cellular communication module).

In response to the occurrence of RBD, the at least one processor 210 may alert the user of the RBD via the user interface 240.

The at least one processor 210 may control the communicator 230 to transmit the sleep state information and the RBD information to the server device 3.

The communicator 230 may include a first communication module for establishing communication with the hub device 1, a second communication module for establishing communication with the server device 3, and a third communication module for establishing communication with the medical institution. Accordingly, the communicator 230 may communicate with the hub device 1 in a first communication scheme, and communicate with the medical institution while communicating with the server device 3 in a second communication scheme.

In an embodiment of the disclosure, the sleep state information and the RBD information may be obtained after the data collected from the plurality of sensors 5 is primarily processed by the hub device 1 and secondarily processed by the user device 2, and the sleep state information and the RBD information may be transmitted to the server device 3 in real time.

In an embodiment of the disclosure, the data collected from the plurality of sensors 5 and associated with the user's privacy may not be transmitted directly to the server device 3.

The user device 2 may include the user interface 240 for communication with the user.

In various embodiments, the sleep state information and the RBD information may also be obtained after the hub device 1 primarily processes the data collected from the plurality of sensors 5 and secondarily processes the primarily processed data.

In various embodiments, the sleep state information and the RBD information may also be obtained after the hub device 1 primarily processes the data collected from the plurality of sensors 5 and transmits the primarily processed data to the server device 3 and the server secondarily processes the primarily processed data.

In various embodiments, the sleep state information and the RBD information may also be obtained, after the hub device 1 transmits the data collected from the plurality of sensors 5 to the user device 2 and the user device 2 primarily processes the data received from the hub device 1 and secondarily processes the primarily processed data.

In various embodiments, the sleep state information and the RBD information may also be obtained, after the hub device 1 transmits the data collected from the plurality of sensors 5 to the server device 3 and the server device 3 primarily processes the data received from the hub device 1 and secondarily processes the primarily processed data.

The user interface 240 may obtain a user input. The user interface 240 may provide various information about operations of the user device 2. The user interface 240 may include an input interface and an output interface.

The input interface may convert sensory information received from the user into an electrical signal. The electrical signal may correspond to the user input. The user input may include various commands. The input interface may transmit the electrical signal (voltage or current) corresponding to the user input to the at least one processor 210.

The input interface may include various input devices to convert tactile information into an electrical signal. For example, the input interface may be implemented with a physical button or a touch screen. The input interface may include a microphone to convert auditory information into an electrical signal.

The input interface may receive a user input to execute the sleep management application.

The output interface may output information associated with operations of the user device 2. The output interface may display information input by the user or information to be provided for the user in various screens. The output interface may display information about an operation of the user device 2 in at least one of an image or text. For example, the output interface may output an interface of the sleep management application. Furthermore, the output interface may display a Graphic User Interface (GUI) that enables the user device 2 to be controlled. In other words, the output interface may display a user interface element (UI element) such as an icon.

The output interface may output an interface corresponding to the sleep management application.

For example, the output interface may include a Liquid Crystal Display (LCD) panel, a Light Emitting Diode (LED) panel, an organic LED (OLED) panel, or a micro LED panel. The output interface may include a touch display that serves as an input device as well.

The output interface and the input interface may be provided separately or in a single device (e.g., the touch display).

The server device 3 may receive, from the user device 2, data obtained by the user device 2 by wireless communication.

The data obtained by the user device 2 may include the sleep state information and the RBD information.

In an embodiment, the server device 3 may include at least one memory 320 for storing a program for processing the sleep state information and the RBD information received from the user device 2, and at least one processor 310 that may process the sleep state information and the RBD information received from the user device 2 based on the program stored in the at least one memory 320.

The at least one memory 320 may store the sleep state information and the RBD information received from the user device 2.

The at least one memory 320 may store a program for generating sleep summary information based on the sleep state information and the RBD information received from the user device 2.

The at least one processor 310 may generate the sleep summary information based on the sleep state information and the RBD information accumulated and stored in the at least one memory 320.

The at least one memory 320 may store a program for controlling the home appliance 4 based on at least one of the sleep state information or the RBD information received from the user device 2.

The at least one processor 310 may control the home appliance 4 based on at least one of the sleep state information or the RBD information received from the user device 2.

In various embodiments, the program for generating the sleep summary information based on at least one of the sleep state information or the RBD information, and the program for controlling the home appliance 4 based on at least one of the sleep state information or the RBD information may be stored in different servers.

For example, a first server included in the server device 3 may store the program for generating the sleep summary information based on at least one of the sleep state information or the RBD information, and a second server included in the server device 3 may store the program for controlling the home appliance 4 based on at least one of the sleep state information or the RBD information.

The at least one memory 320 may store the sleep summary information generated based on at least one of the sleep state information or the RBD information.

In an embodiment, the server device 3 may include a communicator 330 including at least one communication module for establishing communication with the hub device 1, the user device 2, the home appliance 4 and/or the smart sensor device including at least some of the plurality of sensors 5.

The server device 3 may receive the sleep state information and the RBD information from the user device 2 through the communicator 330.

The server device 3 may transmit a control command to the home appliance 4 through the communicator 330.

The server device 3 may transmit the sleep summary information and the RBD information to the user device 2 through the communicator 330.

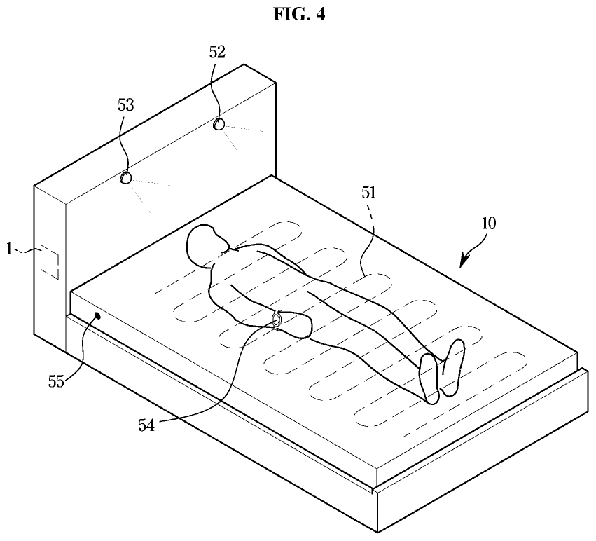

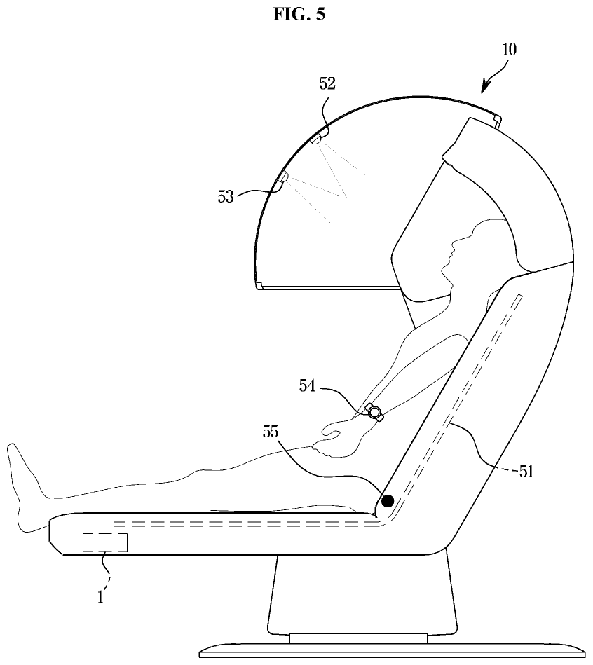

FIG. 4 and FIG. 5 illustrate an example of a plurality of sensors of a sleep management system according to an embodiment.

Referring to FIG. 4 and FIG. 5, in an embodiment, some of the plurality of sensors 5 (e.g., the first sensor 51, the second sensor 52 and/or the third sensor 53) may be installed on a piece of furniture 10, and the others (e.g., the fourth sensor 54 and/or the fifth sensor 55) among the plurality of sensors 5 may be installed in the smart sensor device (e.g., a smart watch, a smart ring, etc.).

At least some of the plurality of sensors 5 may be disposed in the furniture 10 on which the user may sit or lie.

The furniture 10 on which the user may sit or lie may include, for example, a bed, a chair and/or a sofa, or any furniture having the form that allows the user to sit or lie thereon may be used as the furniture 10 without limitation.

In an embodiment, the furniture 10 such as a bed, a chair and/or a sofa may include an actuator that may change the user's posture by changing its structure, and/or a vibration element that may transmit vibration to the user.

The first sensor 51 may include a pressure sensor. The pressure sensor may include a piezoelectric element that generates an electrical signal corresponding to displacement created by the pressure.

The first sensor 51 may be installed in a location where the pressure generated by the user's body (e.g., the whole body) when the user lies or sits may be measured.

For example, in a case where the furniture 10 corresponds to a bed, the first sensor 51 may be disposed in a mattress where pressure from the user's body occurs. The mattress may include a cover having a polygonal or circular flat shape, defining the exterior and having an accommodation space, and a pad arranged in the accommodation space of the cover and including the first sensor 51. The mattress may be placed on the floor, a chair, a sofa or a bed.

The mattress may further include springs and/or sponge. The spring and/or the sponge may be arranged in the accommodation space of the cover.

The structure (e.g., length, layout, etc.) of the first sensor 51 may vary by the size of the mattress.

In another example, in a case where the furniture 10 corresponds to a chair, the first sensor 51 may be disposed in a seating portion, a backrest portion, a headrest and/or a leg portion where pressure from the user's body occurs.

The seating portion may include a portion coming into contact with the user's buttocks, the backrest portion may include a portion coming into contact with the user's back, the headrest may include a portion coming into contact with the user's head, and the leg portion may include a portion coming into contact with the user's legs.

The location of the first sensor 51 is not limited to the example shown in FIG. 4 and FIG. 5, and the first sensor 51 may be installed at various locations where the pressure generated by the body of the user who lies or sits on the furniture 10 may be measured.

The first sensor 51 may measure the pressure generated by the user who lies or sits on the furniture 10. For example, the first sensor 51 may measure a distribution of the pressure that occurs by the user who lies or sits on the furniture 10. The first sensor 51 may obtain pressure data corresponding to the pressure generated by the user who lies or sits on the furniture 10.

The second sensor 52 may include an Ultra-Wide Band (UWB) sensor. The UWB sensor may include a UWB signal transmitter for transmitting a UWB signal and a UWB signal receiver for receiving a UWB signal reflected by the user's body.

The second sensor 52 may have a detection region facing the body (e.g., torso) of the user who lies or sits on the furniture 10. The second sensor 52 may have a detection region that may detect displacement of the body caused by the user's breathing. The second sensor 52 may be disposed on the frame of the furniture 10 to have the detection region facing the body (e.g., torso) of the user, but the location of the second sensor 52 is not limited thereto.

For example, the second sensor 52 may have a detection region facing a portion of the body of the user who lies or sits on the furniture 10.

For example, in a case where the furniture 10 corresponds to a bed, the second sensor 52 may have a detection region facing a center portion of the bed.

In another example, in a case where the furniture 10 corresponds to a chair, the second sensor 52 may have a detection region facing a backrest portion of the chair.

The second sensor 52 may transmit a UWB signal to the body of the user and receive the UWB signal reflected from the body of the user.

The second sensor 52 may measure displacement of the user's body based on the UWB signal reflected from the user's body. For example, the second sensor 52 may measure displacement of the user's body based on a Time Of Flight (ToF) of the UWB signal. In another example, the second sensor 52 may use the Doppler effect to measure the displacement of the user's body according to a change in wavelength (and frequency) of the UWB signal.

In other words, the second sensor 52 may obtain displacement data corresponding to the displacement of the body that changes according to the user's breathing.

The third sensor 53 may include a radar sensor. The third sensor 53 may include a radar signal transmitter for transmitting a radar signal (e.g., millimeter waves or mmWave signal) and a radar signal receiver for receiving the radar signal (e.g., mmWave signal) reflected from the user's body.

A frequency band (e.g., 28 GHz) of the radar signal output from the third sensor 53 may be higher than a frequency band (e.g., 6.0 to 8.8 GHZ) of the UWB signal output from the second sensor 52.

A bandwidth of the radar signal output from the third sensor 53 may be narrower than a bandwidth of the UWB signal output from the second sensor 52.

The third sensor 3 may have a detection region facing the body (e.g., face) of the user who lies or sits on the furniture 10. The third sensor 53 may have a detection region that may detect a movement of the user's eyes. The third sensor 53 may be disposed on the frame of the furniture 10 to have the detection region facing the user's body (e.g., torso), but the location of the third sensor 53 is not limited thereto.

The third sensor 53 may have a detection region facing a portion of the body of the user who lies or sits on the furniture 10.

For example, in a case where the furniture 10 corresponds to a bed, the third sensor 53 may have a detection region facing a head area of the bed.

In another example, in a case where the furniture 10 corresponds to a chair, the third sensor 53 may have a detection region facing the headrest of the chair.

The third sensor 53 may transmit a radar signal (millimeter waves or mm Wave signal) to the user's body, and receive an mm Wave signal reflected from the user's body.

The third sensor 53 may measure the movement of the user's eyes based on the mm Wave reflected from the user's eyes.

In other words, the third sensor 53 may obtain eye-movement data corresponding to the movement of the user's eyes.

The fourth sensor 54 may include a PPG sensor and/or an electrocardiogram sensor. The PPG sensor and/or the electrocardiogram sensor may include a light source for irradiating light and a light receiver for receiving light reflected from the user's body.

The fourth sensor 54 may have a detection region facing the body of the user who lies or sits on the furniture 10.

The fourth sensor 54 may be disposed in the smart sensor device (e.g., a smart watch, a smart ring, etc.) that may be worn by the user.

The fourth sensor 54 may operate in a non-invasive manner by irradiating light to a portion (e.g., a wrist) of the user's body and receiving light reflected from the user's body.

The fourth sensor 54 may measure a heart rate of the user, an oxygen saturation level in the user's blood, and an electrocardiogram (ECG) of the user based on the intensity of the light reflected from the user's body.

A portion of the light irradiated to a portion of the user's body may be absorbed in a blood vessel, and the user's heart rate, the oxygen saturation level in the user's blood, or the user's ECG may be measured according to the light absorption rate and patterns of the absorbed light.

The fifth sensor 55 may include an acceleration sensor. The acceleration sensor may include a Microelectromechanical System (MEMS) sensor, a 3-axis acceleration sensor and/or a 6-axis acceleration sensor.

In various embodiments, like the fourth sensor 54, the fifth sensor 55 may be disposed in the smart sensor device (e.g., a smart watch, a smart ring, etc.) that may be worn by the user.

In an embodiment, the fifth sensor 55 may be installed in the furniture 10.

The fifth sensor 55 may obtain acceleration data corresponding to the movement of the user's body.

The hub device 1 may be installed on the furniture 10 or at a location adjacent to the furniture 10 and may be wiredly connected to some of the plurality of sensors 5. Furthermore, the hub device 1 may perform wireless communication with the smart sensor device (e.g., a wearable device) worn by the user.

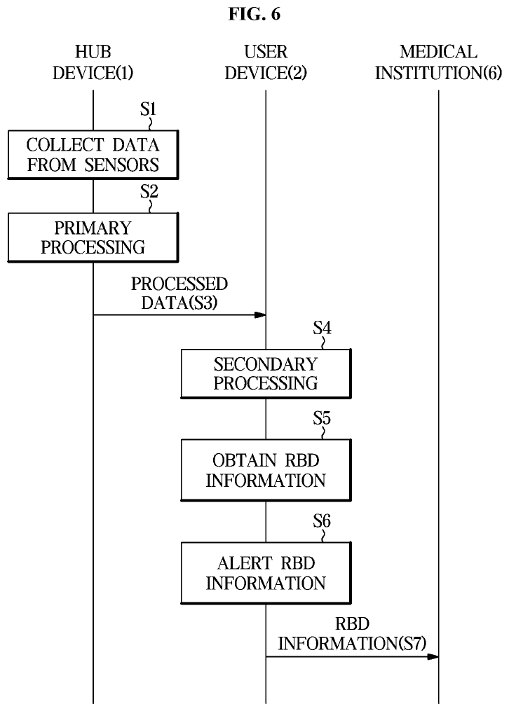

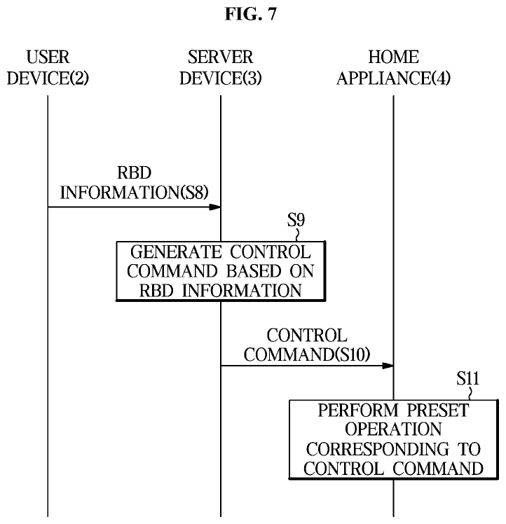

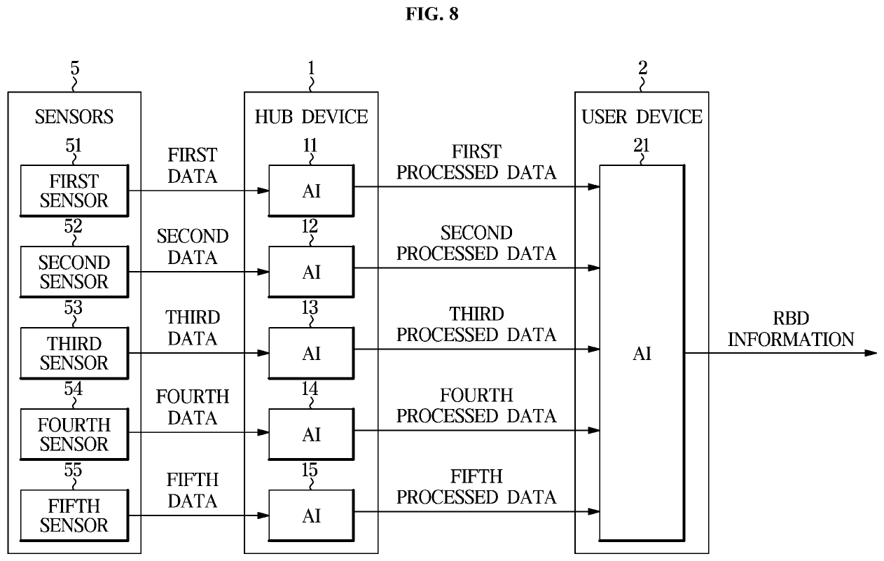

FIG. 6 is a flowchart illustrating an example of a sleep management method according to an embodiment. FIG. 7 is a flowchart illustrating another example of a sleep management method according to an embodiment. FIG. 8 is a diagram for illustrating a procedure for processing data collected from a plurality of sensors of a sleep management system according to an embodiment.

Referring to FIG. 6, FIG. 7 and FIG. 8, the hub device 1 may collect data from the plurality of sensors 5 (S1).

In a case where the plurality of sensors 5 are maintained in an active state, there may be a large consumption of power supplied to the plurality of sensors 5. The plurality of sensors 5 maintained in the active state may include the plurality of sensors 5 maintained in a state of obtaining sensor data by receiving power.

In an embodiment, the hub device 1 may maintain at least some of the plurality of sensors 5 in an inactive state, and switch the plurality of sensors 5 to the active state based on a preset condition being satisfied.

For example, the hub device 1 may determine whether a user is present on the furniture 10 based on processing of the data collected from the plurality of sensors 5. The presence of the user on the furniture 10 may include the user lying or sitting on the furniture 10.

The hub device 1 may switch the plurality of sensors 5 to a standby state based on determining that a user is not present on the furniture 10.

For example, the hub device 1 may deactivate remaining sensors other than the first sensor 51 and operate the first sensor 51 in a low power mode. The deactivating of the sensor may include blocking power supplied to the sensor.

The operating of the sensor in the low power mode may include setting an operation period (e.g., a data collection period) of the sensor to be longer.

The hub device 1 may determine whether the user is present on the furniture 10 based on processing of the data collected by the sensor (e.g., the first sensor 51) operating in the low power mode among the plurality of sensors 5.

The hub device 1 may wake up the plurality of sensors 5 based on determining that the user is present on the furniture 10.

Based on activation of the plurality of sensors 5, the collected data may be transmitted to the hub device 1.

The first sensor 51 may transmit first data to the hub device 1, the second sensor 52 may transmit second data to the hub device 1, the third sensor 53 may transmit third data to the hub device 1, the fourth sensor 54 may transmit fourth data to the hub device 1, and the fifth sensor 55 may transmit fifth data to the hub device 1.

In an embodiment, at least some of the plurality of sensors 5 may transmit the sensor data to the hub device 1 by wired communication, and the others of the plurality of sensors 5 may transmit the sensor data to the hub device 1 by wireless communication.

The hub device 1 may primarily process the data collected from the plurality of sensors 5 (S2). To this end, the hub device 1 may be equipped with a machine learning model.

The data collected from the plurality of sensors 5 may be processed by the machine learning models 11, 12, 13, 14 and 15 deployed on the hub device 1.

The first machine learning model 11 for extracting a feature from the first data collected from the first sensor 51, the second machine learning model 12 for extracting a feature from the second data collected from the second sensor 52, the third machine learning model 13 for extracting a feature from the third data collected from the third sensor 53, the fourth machine learning model 14 for extracting a feature from the fourth data collected from the fourth sensor 54, and the fifth machine learning model 15 for extracting a feature from the fifth data collected from the fifth sensor 55 may be deployed on the hub device 1.

The first machine learning model 11 may be pre-trained to extract a feature from pressure data collected by the pressure sensor. The second machine learning model 12 may be pre-trained to extract a feature from displacement data collected by the UWB sensor. The third machine learning model 13 may be pre-trained to extract a feature from eye-movement data collected by the radar sensor. The fourth machine learning model 14 may be pre-trained to extract a feature from heart rate data, oxygen saturation data and/or ECG data collected by the PPG sensor and/or the ECG sensor. The fifth machine learning model 15 may be pre-trained to extract a feature from acceleration data collected by the acceleration sensor.

The first machine learning model 11 may use the first data collected by the first sensor 51 as input data to output first processed data as output data. The first processed data may include, for example, information about the user's posture, respiration rate and heart rate inferred by the first data. In another example, the first processed data may include at least one of information about the user's sleep stage or information about the user's body movement inferred by the first data.

The second machine learning model 12 may use the second data collected by the second sensor 52 as input data to output second processed data as output data. The second processed data may include, for example, information about a respiration rate and/or a heart rate inferred by the second data. In another example, the second processed data may include at least one of information about the user's sleep stage or information about the user's body movement inferred by the second data.

The third machine learning model 13 may use the third data collected by the third sensor 53 as input data to output third processed data as output data. The third processed data may include, for example, information about an eye movement inferred by the third data. In another example, the third processed data may include at least one of information about the user's sleep stage, information about the user's eye movement, or information about the user's body movement inferred by the third data.

The fourth machine learning model 14 may use the fourth data collected by the fourth sensor 54 as input data to output fourth processed data as output data. The fourth processed data may include, for example, information about a heart rate, an oxygen saturation level and/or an ECG inferred by the fourth data. In another example, the fourth processed data may include information about the user's sleep stage or information about the user's body movement inferred by the fourth data.

The fifth machine learning model 15 may use the fifth data collected by the fifth sensor 55 as input data to output fifth processed data as output data. The fifth processed data may include, for example, information about a movement inferred by the fifth data. In another example, the fifth processed data may include at least one of information about the user's sleep stage or information about the user's body movement inferred by the fifth data.

In the disclosure, the hub device 1 may primarily process the data collected from the plurality of sensors 5, thereby transmitting the data collected from the plurality of sensors 5 to the user device 2 in a communication scheme with low data transfer capacity.

The hub device 1 may process the data collected from the plurality of sensors 5, and transmit the processed data to the user device 2 (S3).

The hub device 1 may transmit the processed data to the user device 2 by wireless communication. In an embodiment, the communicator 130 of the hub device 1 may include a first communication module for receiving data collected from some (e.g., the fourth sensor 54) of the plurality of sensors 5 in a first wireless communication scheme, and a second communication module for transmitting the processed data to the user device 2 in a second wireless communication scheme.

The first wireless communication scheme and the second wireless communication scheme may be the same or different from each other.

In an embodiment of the disclosure, because the hub device 1 is equipped with both the first communication module for receiving data from some of the plurality of sensors 5 and the second communication module for communicating with the user device 2, the hub device 1 may communicate with the plurality of sensors 5 and the user device 2 simultaneously.

The user device 2 may process the processed data received from the hub device 1 (S4). To this end, the user device 2 may be equipped with a machine learning model.

The processed data received from the hub device 1 may be processed by a machine learning model 21 deployed on the user device 2.

The machine learning model 21 deployed on the user device 2 may include an artificial neural network (deep neural network) model with several layers (e.g., an input layer, a hidden layer, and an output layer). The machine learning model 21 deployed on the user device 2 may have a perceptron structure that receives multiple signals and outputs one signal. The machine learning model 21 deployed on the user device 2 may be trained to estimate the user's sleep state based on the processed data processed by the hub device 1.

The machine learning model 21 deployed on the user device 2 may use the processed data output by the machine learning model of the hub device 1 as input data to output sleep state information associated with the user's sleep state as output data.

The machine learning model 21 deployed on the user device 2 may use the first processed data, the second processed data, the third processed data, the fourth processed data and/or the fifth processed data as input data to output the sleep state information.

The sleep state information output by the machine learning model 21 deployed on the user device 2 may include at least one of information about the user's sleep stage or information about the user's body movement.

The user device 2 may obtain RBD information associated with the user's RBD based on the user's sleep stage and the user's body movement (S5). In response to the user's sleep stage being a REM sleep stage and the user's body movement being greater a preset movement, the user device 2 may determine that RBD occurs.

The user device 2 may store the RBD information of the user in the at least one memory 220.

In the disclosure, because the data primarily output by the machine learning models 11, 12, 13, 14 and 15 deployed on the hub device 1 is secondarily input to the machine learning model 21 deployed on the user device 2 to output the sleep state information, the user's sleep state may be accurately estimated. In the disclosure, a large amount of data may be processed in stages, the user's sleep state may be accurately estimated. Accordingly, whether RBD occurs may be determined based on the user's exact sleep state, thereby estimating whether RBD occurs more conveniently, effectively, and accurately.

The user device 2 may alert the user of the RBD through the user interface 240 in response to the occurrence of RBD (S6).

The user device 2 may alert the user of the RBD at the time the RBD occurs.

The user device 2 may store the RBD information in the at least one memory 220 at the time the RBD occurs, and then alert the user of the RBD when the user's sleep stage is an awakening stage.

The user device 2 may suggest a hospital diagnosis to the user as RBD occurs.

In response to the occurrence of RBD, the user device 2 may transmit the RBD information to a medical institution 6, registered in conjunction with the user device 2, via the communicator 230 (e.g., cellular communication module) to notify the external medical institution 6 of the occurrence of RBD (S7).

The user device 2 may notify a hospital (medical institution) of the occurrence of the user's RBD as the RBD occurs.

Upon receiving the RBD information, the medical institution 6 may take measures to prevent safety accidents for the user, thereby protecting the user safely.

The user device 2 may transmit the RBD information to the server device 3 (S8). In this instance, the user device 2 may transmit the sleep state information and the RBD information to the server device 3.

In the disclosure, instead of transmitting data directly involved with the user's privacy to the server device 3, only the sleep state information may be transmitted to the server device 3, thereby gaining the user's agreement on the data collection more easily.

The server device 3 may generate a control command to control the home appliance 4 based on the RBD information (S9). In this case, the home appliance 4 may include at least one home appliance 4 registered in conjunction with the user device 2. The server device 3 may store and/or manage the user account, and register the user device 2 and the home appliance 4 by associating them with the user account.

The at least one home appliance 4 registered in conjunction with the user device 2 may include the home appliance 4 registered with the user account with which the user device 2 is registered.

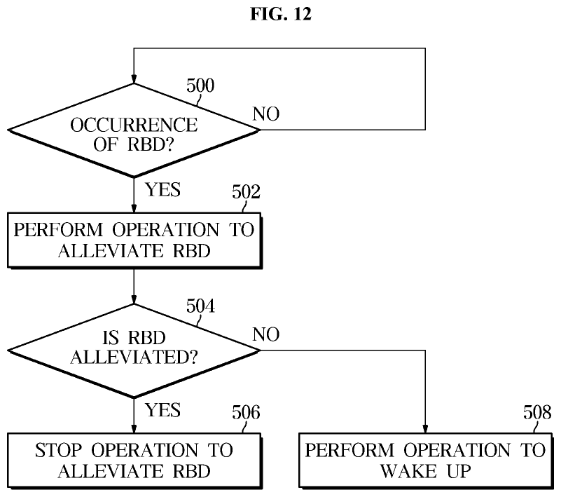

The procedure S9 for generating the control command to control the home appliance 4 based on the RBD information will be described in detail later with reference to FIG. 12 to FIG. 15. The server device 3 may transmit the control command to control the home appliance 4 to the home appliance 4 (S10).

In various embodiments, the procedure S9 for generating the control command to control the home appliance 4 based on the RBD information and the procedure S10 for transmitting the control command to control the home appliance 4 to the home appliance 4 may be performed by the user device 2 as well.

The home appliance 4 may perform a preset operation corresponding to the control command received from the server device 3 (S11).

In various embodiments, the server device 3 may control the home appliance 4 to perform an operation to alert the user of the RBD in response to the occurrence of RBD. The server device 3 may alert the user of the RBD through the home appliance 4 at the time the RBD occurs or when the user's sleep stage is the awakening stage. The server device 3 may suggest a hospital diagnosis to the user as the RBD occurs.

In various embodiments, in response to the occurrence of RBD, the server device 3 may transmit the RBD information to a medical institution, registered in conjunction with the user device 2, by wireless communication. The server device 3 may notify the hospital of the occurrence of the user's RBD as the RBD occurs.

The machine learning model stored in the user device 2 may be updated by an external server. To this end, in various embodiments, data output by the machine learning models 11, 12, 13, 14 and 15 deployed on the hub device 1 may be transmitted to the server device 3.

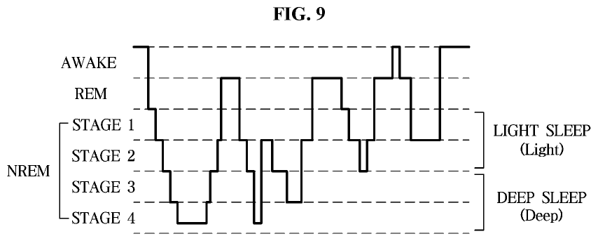

FIG. 9 is a diagram for illustrating sleep stages.

Sleep stages of humans may be classified into an awakening stage, a Rapid Eye Movement (REM) sleep stage, and a Non-REM (NREM) sleep stage.

The awakening stage corresponds to a stage in which a person is awake.

The REM sleep stage is a stage of rapid eye movement sleep, which corresponds to a light sleep close to being awake, and is a sleep stage characterized by rapid eye movements. The REM sleep stage occurs after the NREM sleep stage and may first occur approximately 90 minutes after falling asleep. During the REM sleep stage, heart rate and brain wave patterns are similar to those in the awakening stage, muscle tone decreases, and breathing rate becomes rapid and irregular.

The NREM sleep stage is a stage of non-rapid eye movement sleep, and is characterized by slow eye movements, low heart rate, low breathing rate, and muscle relaxation.

The NREM sleep stage may be divided into stage 1 (N1 stage), stage 2 (N2 stage), stage 3 (N3 stage) and stage 4 (N4 stage). In the NREM sleep stage, stage 1 (N1 stage) and stage 2 (N2 stage) may be classified as a light sleep stage, and stage 3 (N3 stage) and stage 4 (N4 stage) may be classified as a deep sleep stage.

In another example, the NREM sleep stage may also be divided into stage 1 (N1 stage), stage 2 (N2 stage) and stage 3 (N3 stage).

In the NREM sleep stage, brainwave activity gradually slows down and the physiological function decreases.

The N1 stage is the most borderline phase in the sleep state, indicating a procedure in which human body slowly falls asleep. The N1 stage occurs before falling into a deep sleep, and slow eye movements may be observed.

The N2 stage is a phase that goes into a deeper sleep from the borderline sleep phase.

The N3 stage is an early stage of deep sleep with loss of muscle tone and little body movement.

The N4 stage corresponds to a deep sleep stage where it may be difficult to be awake.

Sleep in the N3 to N4 stages, which is slow-wave sleep, is most common in the first third of the sleep cycle, while REM sleep is observed in the last third.

It is common for the first sleep cycle to begin when a person starts to fall asleep. In the first sleep cycle, the person enters into the NREM sleep stage from the awakening stage, goes through the N1, N2, N3 and N4 stages, and goes back to the N3, N2, N1, and REM sleep stages.

Subsequently, in the second sleep cycle, the person goes through the N1, N2, N3 and N4 stages and goes back to the N3 and N2 stages.

Subsequently, in the third sleep cycle, the person goes through the N3 stage and goes back to the N2, N1 and REM sleep stages.

Subsequently, in the fourth sleep cycle, the person enters back into the N1 and REM sleep stages after going through the N1 and N2 stages.

The person then naturally wakes up while going through the N1, N2 and REM sleep stages.

RBD may occur during REM sleep.

Based on the time from falling asleep to waking up, REM sleep occurs 4 to 5 times at approximately 90-minute intervals and generally lasts for from 10 minutes to 30 minutes.

RBD may occur more than 2 hours after falling asleep and be characterized by a behavior of acting out a dream and a behavior of waking up immediately after acting out the dream.

RBD may also be characterized by increased muscle tone during REM sleep and excessive movements and abnormal behavior associated with dreams.

Patients with RBD may experience unpleasant and violent dreams, such as fighting with someone, being attacked, or being chased, and act out the dream, causing them to fall out of bed and hurt themselves, or to injure a sleeping partner.

RBD may be accompanied by aggressive body movements during the REM sleep stage. Accordingly, a user's occurrence of RBD may be diagnosed by monitoring the user's body movements during the REM sleep stage.

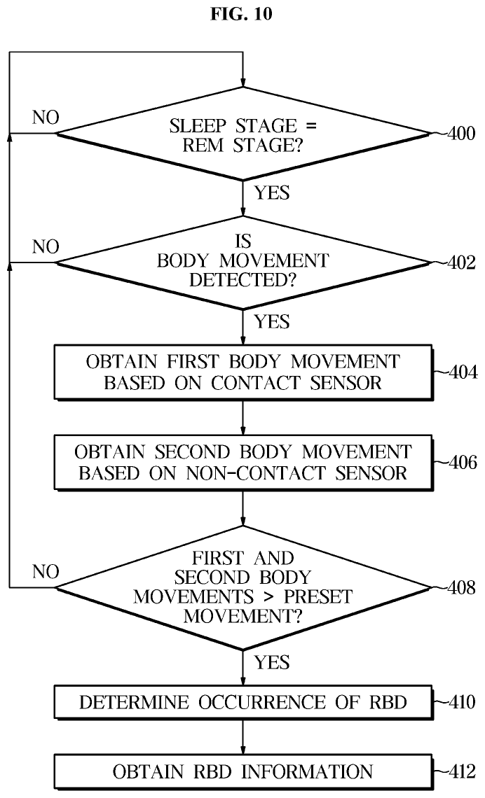

FIG. 10 illustrates an example of obtaining RBD information of a user in a sleep management method according to an embodiment.

Referring to FIG. 10, the user device 2 may determine whether a sleep stage of a user is a REM sleep stage (400).

The user device 2 may continuously determine the user's sleep stage after the user falls asleep to determine whether the user's sleep stage is a REM sleep stage.

The user device 2 may input processed data, obtained based on processing of data collected from the plurality of sensors 5, to a machine learning model, thereby obtaining sleep state information associated with the user's sleep stage and the user's body movement.

The user device 2 may determine the user's current sleep stage based on the sleep state information.

When the user's sleep stage changes from a NREM sleep stage to the REM sleep stage, the user device 2 may determine that the user has entered REM sleep.

In response to the user's sleep stage being the REM sleep stage (Yes in operation 400), the user device 2 may determine whether the user's body movement is detected (402).

The user device 2 may determine the user's current body movement based on the sleep state information.

In response to detection of the user's body movement (Yes in operation 402), the user device 2 may obtain information about a first body movement of the user based on a contact sensor (404).

The user device 2 may obtain the information about the first body movement of the user by inputting at least one processed data corresponding to data collected by the contact sensor from among first processed data, second processed data, third processed data, fourth processed data, and/or fifth processed data to a machine learning model. In this instance, the first processed data, the second processed data, the third processed data, the fourth processed data, and the fifth processed data may be obtained based on processing of first data, second data, third data, fourth data, and fifth data collected from the plurality of sensors 5. For example, the user device 2 may obtain the information about the first body movement of the user by inputting processed data, obtained by processing of data collected from at least one of a pressure sensor or an acceleration sensor corresponding to the contact sensor, to the machine learning model.

The user device 2 may obtain information about a second body movement of the user based on a non-contact sensor (406).

The user device 2 may obtain the information about the second body movement of the user by inputting at least one processed data corresponding to data collected by the non-contact sensor from among the first processed data, the second processed data, the third processed data, the fourth processed data, and/or the fifth processed data to the machine learning model. In this instance, the first processed data, the second processed data, the third processed data, the fourth processed data, and the fifth processed data may be obtained based on processing of the first data, the second data, the third data, the fourth data, and the fifth data collected from the plurality of sensors 5. For example, the user device 2 may obtain the information about the second body movement of the user by inputting processed data, obtained by processing of data collected from a UWB sensor corresponding to the non-contact sensor, to the machine learning model.

The user device 2 may determine whether both the first body movement and the second body movement are greater than a preset movement (408). In this instance, the user device 2 may compare the first body movement and the second body movement with the same preset movement. In addition, the user device 2 may compare the first body movement and the second body movement with different preset movements. For example, the user device 2 may compare the first body movement with a first preset movement to determine whether the first body movement is greater than the first preset movement, and may compare the second body movement with a second preset movement to determine whether the second body movement is greater than the second preset movement.

In response to both the first body movement and the second body movement being greater than the preset movement (Yes in operation 408), the user device 2 may determine that RBD occurs (410).

The user device 2 may count the number of times that both the first body movement and the second body movement are greater than the preset movement during the REM sleep stage, and in response to the counted number of times being greater than a preset number of times, the user device 2 may determine that RBD occurs.

The user device 2 may obtain RBD information associated with the user's RBD (412).

The RBD information may include at least one of information about whether RBD occurs, information about a degree of RBD, or information about an occurrence time of RBD.

The user device 2 may determine whether RBD occurs, the degree of RBD, the occurrence time of RBD, and the like, by analyzing body movement patterns such as intensity, size, and motion of the first body movement and the second body movement. For example, during the REM sleep stage, in a case where a dangerous dream-related body movement or a body movement acting out the user's dream occurs, the user device 2 may determine that RBD occurs.

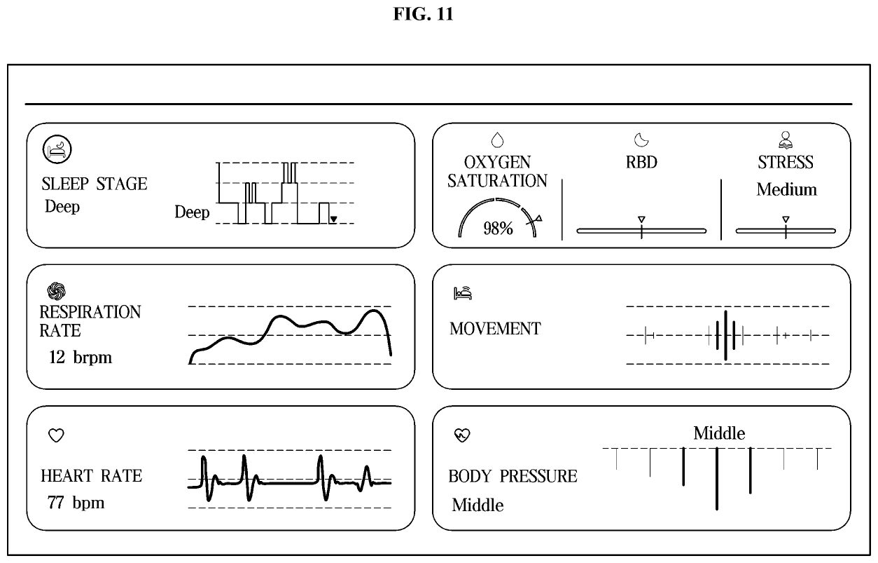

FIG. 11 illustrates an example of sleep state information and RBD information derived by a sleep management system according to an embodiment.

Referring to FIG. 11, sleep state information and RBD information may be derived by the sleep management system in real time.

In various embodiment, the user device 2 (or the display device 41) may output the sleep state information and/or the RBD information through an output interface (e.g., a display).

The sleep state information may include information about a sleep stage, a body movement, an oxygen saturation, a stress index, a respiration rate, a heart rate, and/or body pressure.

The information about the sleep stage may include information about a current sleep stage of the user and/or information about the user's sleep stages over time.

The information about the user's current sleep stage may include information indicating which one of awakening stage, REM sleep stage, or the stage 1, stage 2 or stage 3 of NREM sleep stage the current sleep stage of the user corresponds to.

The information about the user's sleep stages over time may include information about changes in sleep stage to the current time from a time preset by the user with the user device 2, a time when the user lies or sits on the furniture 10 to sleep, a time when the user falls asleep, and/or a time when the user runs the sleep management application.

In a case where the information about the user's sleep stages over time is output by the user device 2 (or the display device 41), the information about the user's sleep stages over time may be represented in the form of a graph having the X-axis indicating time and the Y-axis indicating the user's sleep stages.

The information about oxygen saturation may include information about current oxygen saturation of the user and/or information about the user's oxygen saturation over time.

In a case where the information about oxygen saturation is output by the user device 2 (or the display device 41), numerical values of the oxygen saturation may be output in percentage. Furthermore, in a case where the information about oxygen saturation is output by the user device 2 (or the display device 41), whether the oxygen saturation of the user is normal according to medical standards may be displayed.

The information about stress index may include information about a stress level of the user. In a case where the information about stress index is output by the user device 2 (or the display device 41), the user's stress index may be output in a numerical value or in the form of a comparative word (e.g., high, medium, and low) with an indication of whether the user's stress index is normal according to the medical standard.

The information about a respiration rate may include information about a current respiration rate of the user and/or information about the user's respiration rates over time.

In a case where the information about the current respiration rate is output by the user device 2 (or the display device 41), the user's respiration rate may be output in a numerical value with an indication of whether the user's respiration rate is normal according to the medical standard.

In a case where the information about the user's respiration rates over time is output by the user device 2 (or the display device 41), the information about the user's respiration rates over time may be represented in the form of a graph having the X-axis indicating time and the Y-axis indicating the user's respiration rates.

The information about movement may include information about a degree of the user's movement and/or information about the user's movement degrees over time.

In a case where the information about the current movement degree is output by the user device 2 (or the display device 41), the degree of user's movement may be output in a numerical value or in the form of a comparative word (e.g., high, medium, and low).

In a case where the information about movement degrees over time is output by the user device 2 (or the display device 41), the information about the user's movement degrees over time may be represented in the form of a graph having the X-axis indicating time and the Y-axis indicating the user's movement degrees.

The information about movement may include information about a body movement and information about eye movement.

The information about a heart rate may include information about a current heart rate of the user and/or information about the user's heart rates over time.

In a case where the information about the current heart rate is output by the user device 2 (or the display device 41), the user's heart rate may be output in a numerical value with an indication of whether the user's heart rate is normal according to the medical standard.

In a case where the information about the user's heart rates over time is output by the user device 2 (or the display device 41), the information about the user's heart rates over time may be represented in the form of a graph having the X-axis indicating time and the Y-axis indicating pulses corresponding to the user's heart rates.

The information about body pressure may include information about the user's body posture. The information about body posture may include information about whether the user's posture corresponds to lying on one's back, lying on the left side, lying on the right side, or lying on one's stomach, sitting up, etc.

In a case where the information about body pressure is output by the user device 2 (or the display device 41), a term representing the user's posture may be used, and the form of a pressure distribution table based on the furniture 10 on which the user lies or sits may be output.

The RBD information of the user may include at least one of information about whether RBD occurs, information about a degree of RBD, or information about an occurrence time of RBD.

In a case where information about a current RBD is output by the user device 2 (or the display device 41), whether RBD occurs, the degree of RBD, the occurrence time of RBD, and the like, may be output as a numerical value or as text.

The sleep state information and/or the RBD information may be provided to the user by the user device 2, or by the home appliance 4 (e.g., display device 41, speaker 46, etc.).

For example, the user may run the sleep management application using the user device 2, and check the sleep state information and/or the RBD information through an interface provided by the sleep management application.

In another example, the server device 3 may control the home appliance 4 (e.g., display device 41, speaker 46, etc.) to provide sleep summary information and/or the RBD information when the user wakes up.

In various embodiments, the user may share the sleep state information and/or the RBD information with other users through the user device 2. For example, the user may run the sleep management application and transmit the sleep state information and/or the RBD information to another user's device through an interface provided by the sleep management application.

FIG. 12 illustrates an example of preset operations following an occurrence of RBD of a user in a sleep management method according to an embodiment.

In an embodiment, the server device 3 may control the home appliance 4 based on RBD information.

Referring to FIG. 12, the server device 3 may determine whether RBD of a user occurs based on RBD information (500).

Based on determining that the RBD occurs (Yes in operation 500), the server device 3 may control the home appliance 4 to perform a preset operation to alleviate the user's RBD (502).

In an embodiment of the disclosure, by controlling the home appliance 4 to perform a preset operation to alleviate the user's RBD, the user's RBD may be alleviated by the operation of the home appliance 4.

The preset operation to alleviate RBD performed by the home appliance 4 is not limited to the above-described example, and may vary depending on the user's setting and the type of home appliance 4.

The server device 3 may determine whether the user's RBD has been alleviated (504). The server device 3 may determine whether the RBD is relieved based on the slowing down of changes in the user's body movement after the preset operation for alleviating the RBD has been performed.

In response to the user's RBD having been alleviated (Yes in operation 504), the server device 3 may control the home appliance 4 to stop the preset operation to alleviate the RBD (506).

In response to the user's RBD not having been alleviated (No in operation 504), the server device 3 may control the home appliance 4 to perform a preset operation to wake up the user (508).

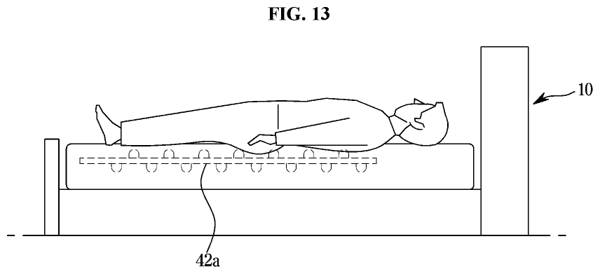

FIG. 13 illustrates an example of a preset operation to alleviate RBD of a user.

Referring to FIG. 13, the server device 3 may control the lighting device 43 to perform a preset operation to alleviate RBD of a user.

For example, the server device 3 may control the lighting device 43 to output light of a preset color (e.g., a color having a color temperature of light of 1000K or less).

The server device 3 may control the air conditioner 45 to perform a preset operation to alleviate the user's RBD.

For example, the server device 3 may control the air conditioner 45 to slightly increase a fan speed to allow the wind output from the air conditioner 45 to reach the user.

The server device 3 may control the furniture control device 42 to perform a preset operation to alleviate the user's RBD. The furniture control device 42 may include a vibration element 42a that may transmit vibration to the user who lies or sits on the furniture 10.

For example, the server device 3 may control the vibration element 42a to output vibration corresponding to the user's heart rate.

In an embodiment of the disclosure, by alleviating the user's RBD, injury to the user or a sleeping partner may be reduced and quality sleep may be induced.

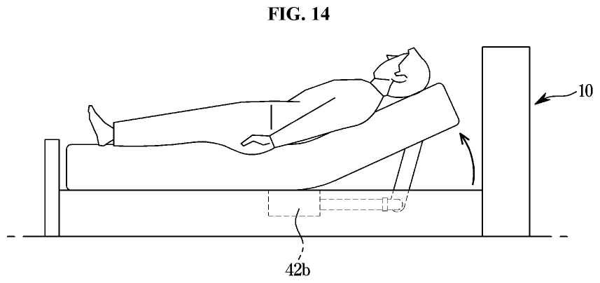

FIG. 14 illustrates another example of a preset operation to alleviate RBD of a user.

Referring to FIG. 14, the server device 3 may control the air conditioner 45 to perform a preset operation to alleviate RBD of a user.

For example, the server device 3 may turn off the air conditioner 45.

The server device 3 may control the furniture control device 42 to perform a preset operation to alleviate the user's RBD. The furniture control device 42 may include an actuator 42b that may change the user's posture by changing the structure of the furniture 10.

For example, the server device 3 may control the actuator 42b to change the structure of the furniture to allow the user's posture to be changed to a preset posture that eases the user's breathing (the preset posture in which the user's head is higher than the torso and leg portions).

In an embodiment of the disclosure, injury to the user or a sleeping partner may be reduced and quality sleep may be induced by alleviating the user's RBD.

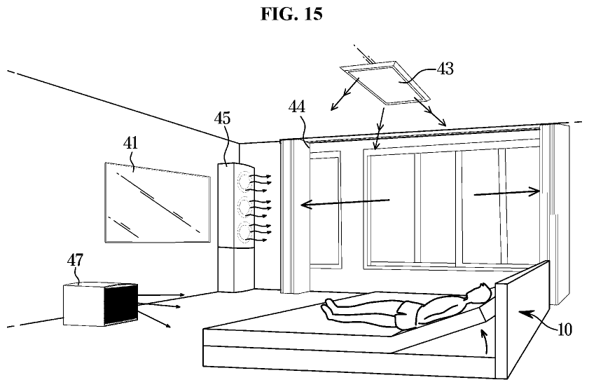

FIG. 15 illustrates an example of a preset operation to induce wakeup of a user.

Referring to FIG. 15, in a case where a user's RBD lasts for a long time or the number of RBD occurrences is significant without alleviating the RBD, the server device 3 may control the display device 41 to perform a preset operation to induce wakeup in order to reduce injury.

For example, the server device 3 may control the display device 41 to gradually increase the brightness of an image output from the display device 41. In another example, the server device 3 may control the display device 41 to play preset music (e.g., music to induce wakeup).

In various embodiments, the server device 3 may control the speaker 46 to play preset music (e.g., music to induce wakeup).

The server device 3 may control the lighting device 43 to perform a preset operation to induce wakeup. For example, the server device 3 may control the lighting device 43 to gradually increase the brightness of light output from the lighting device 43. In another example, the server device 3 may control the lighting device 43 to output light similar to natural light.

The server device 3 may control the automatic curtain open/close device 44 to perform a preset operation to induce wakeup. For example, the server device 3 may control the automatic curtain open/close device 44 to open the curtain.

The server device 3 may control the furniture control device 42 to perform a preset operation to induce wakeup. The furniture control device 42 may include the vibration element 42a that may transmit vibration to the user who lies or sits on the furniture 10, and/or the actuator 42b that may change the posture of the user by changing the structure of the furniture 10.

For example, the server device 3 may control the vibration element 42a to output vibration at preset intervals corresponding to the user's heart rate.

In another example, the server device 3 may control the actuator 42b to change the structure of the furniture to allow the user's posture to be changed to a preset posture for easy wakeup (the preset posture in which the user's upper body is higher than the lower body).

The server device 3 may control the air conditioner 45 and/or the air purifier 47 to perform a preset operation to induce wakeup. For example, the server device 3 may control the air conditioner and/or the air purifier 47 to operate in an awakening mode.

For example, while operating in the awakening mode, the air conditioner 45 and/or the air purifier 47 may control the fan to operate at a high speed to provide a pleasant feeling to the user.

The preset operation to induce wakeup that may be performed by the home appliance 4 is not limited to the above examples, and may be changed according to the user's setting and the type of the home appliance 4.

According to an embodiment of the disclosure, a user's RBD may be screened more conveniently and effectively in daily life without wearing various sensors on the user's body.

According to an embodiment of the disclosure, a user's RBD may be relieved by controlling a home appliance to perform a preset operation to alleviate the user's RBD.

According to an embodiment of the disclosure, by alleviating a user's RBD, the risk of injury to the user or to the user's sleeping partner may be reduced and quality sleep may be induced.

According to an embodiment of the disclosure, the output values of the machine learning model stored in the hub device may be used as the input values of the machine learning model stored in the user device, thereby obtaining accurate data associated with the user's RBD.

According to an embodiment of the disclosure, a sleep management system may include: a plurality of sensors 5 including a first sensor 51, 52, 53, 54 or 55 and a second sensor 51, 52, 53, 54 or 55, the first sensor and the second sensor being configured to collect data of a user; a hub device 1 configured to receive first data collected by the first sensor 51, 52, 53, 54 or 55 and second data collected by the second sensor, obtain first processed data based on processing of the first data, and obtain second processed data based on processing of the second data; and a user device 2 configured to receive the first processed data and the second processed data from the hub device 1, obtain sleep state information associated with a sleep stage and a body movement of the user based on the first processed data and the second processed data, determine whether a Rapid Eye Movement (REM) Sleep Behavior Disorder (RBD) of the user occurs based on the user's body movement when the user's sleep stage is a REM sleep stage, and perform a preset operation in response to an occurrence of the RBD.

The plurality of sensors 5 may include at least two of a pressure sensor, a Ultra-Wideband (UWB) sensor, a radar sensor, a photoplethysmography (PPG) sensor, an electrocardiogram sensor, or an acceleration sensor.

The hub device 1 may be configured to obtain the first processed data by inputting the first data to a first machine learning model 11, 12, 13, 14, or 15, and obtain the second processed data by inputting the second data to a second machine learning model 11, 12, 13, 14, or 15.

The user device may be configured to determine that the RBD occurs, in response to the user's sleep stage being the REM sleep stage and the user's body movement being greater than a preset movement.

The first sensor 51, 52, 53, 54 or 55 may be a contact sensor, and the second sensor 51, 52, 53, 54 or 55 may be a non-contact sensor.

The user device 2 may be configured to: when the user's sleep stage is the REM sleep stage, obtain information about a first body movement of the user by inputting the first processed data, corresponding to the first data collected by the contact sensor, to a machine learning model 21, obtain information about a second body movement of the user by inputting the second processed data, corresponding to the second data collected by the non-contact sensor, to the machine learning model 21, and determine that the RBD occurs in response to both the first body movement and the second body movement being greater than a present movement.

The preset operation may include an operation of alerting the user of the occurrence of the RBD via a user interface 240.

The preset operation may include an operation of notifying a medical institution 6 registered in conjunction with the user device 2 of the occurrence of the RBD.

The sleep management system may further include a server device 3 configured to receive RBD information associated with the RBD from the user device 2.

The server device 3 may be configured to control at least one home appliance 4, registered in conjunction with the user device 2, to perform a preset operation to alleviate the RBD in response to the occurrence of the RBD.

The server device 3 may be configured to control at least one home appliance 4, registered in conjunction with the user device 2, to perform a preset operation to wake up the user in response to the occurrence of the RBD.

The server device 3 may be configured to, in response to the occurrence of the RBD, control at least one home appliance, registered in conjunction with the user device, to perform an operation of alerting the user of the RBD, or perform an operation of transmitting the RBD information to a medical institution 6 registered in conjunction with the user device 2.

According to an embodiment of the disclosure, a sleep management method may include: receiving, by a hub device 1, first data collected by a first sensor 51, 52, 53, 54 or 55 and second data collected by a second sensor 51, 52, 53, 54 or 55; obtaining, by the hub device 1, first processed data based on processing of the first data, and second processed data based on processing of the second data; transmitting, by the hub device 1, the first processed data and the second processed data to a user device 2; obtaining, by the user device 2, sleep state information associated with a sleep stage and a body movement of a user based on the first processed data and the second processed data received from the hub device 1; determining, by the user device 2, whether a Rapid Eye Movement (REM) Sleep Behavior Disorder (RBD) of the user occurs based on the user's body movement when the user's sleep stage is a REM sleep stage; and performing, by the user device 2, a preset operation in response to an occurrence of the RBD.

The determining of whether the RBD occurs may include determining that the RBD occurs in response to the user's sleep stage being the REM sleep stage and the user's body movement being greater than a preset movement.

The first sensor 51, 52, 53, 54 or 55 may be a contact sensor, and the second sensor 51, 52, 53, 54 or 55 may be a non-contact sensor. The determining of whether the RBD occurs may include: when the user's sleep stage is the REM sleep stage, obtaining information about a first body movement of the user by inputting the first processed data, corresponding to the first data collected by the contact sensor, to a machine learning model 21, obtaining information about a second body movement of the user by inputting the second processed data, corresponding to the second data collected by the non-contact sensor, to the machine learning model 21, and determining that the RBD occurs in response to both the first body movement and the second body movement being greater than a present movement.

The performing of the preset operation by the user device 2 may include performing an operation of alerting the user of the occurrence of the RBD via a user interface 240.

The performing of the preset operation by the user device 2 may include performing an operation of notifying a medical institution 6 registered in conjunction with the user device 2 of the occurrence of the RBD.

The sleep management method may further include receiving, by a server device 3, RBD information associated with the RBD from the user device 2.

The sleep management method may further include controlling, by the server device 3, at least one home appliance, registered in conjunction with the user device, to perform a preset operation to alleviate the RBD or to wake up the user in response to the occurrence of the RBD.

The sleep management method may further include, in response to the occurrence of the RBD, controlling, by the server device 3, at least one home appliance to perform an operation of alerting the user of the RBD, or perform an operation of transmitting the RBD information to a medical institution registered in conjunction with the user device.

The disclosed embodiments may be implemented in the form of a recording medium that stores instructions executable by one or more computers. The instructions may be stored in the form of program codes, and when executed by one or more processors processor, the instructions may create a program module to perform operations of the disclosed embodiments. The recording medium may be implemented as a computer-readable recording medium.

The computer-readable recording medium may include all kinds of recording media storing instructions that may be interpreted by one or more computers. For example, the computer-readable recording medium may be one or more among a Read Only Memory (ROM), a Random Access Memory (RAM), a magnetic tape, a magnetic disk, a flash memory, an optical data storage device, etc. That is, the instructions stored on the computer-readable recording medium may be stored on one computer-readable recording medium, or may be distributed among one or more computer-readable recording mediums to implement the method.

The computer-readable storage medium may be provided in the form of a non-transitory storage medium. Here, when a storage medium is referred to as “non-transitory”, it may be understood that the storage medium is tangible and does not include a signal (e.g., an electromagnetic wave), but rather that data is semi-permanently or temporarily stored in the storage medium. For example, a “non-transitory storage medium” may include a buffer in which data is temporarily stored.

According to an embodiment, the method according to the various embodiments disclosed herein may be provided in a computer program product. The computer program product may be traded between a seller and a buyer as a product. The computer program product may be distributed in the form of a machine-readable storage medium (e.g., compact disc read only memory (CD-ROM)), or may be distributed (e.g., download or upload) through an application store (e.g., Play Store™) online or directly between two user devices (e.g., smartphones). In the case of online distribution, at least a portion of the computer program product (e.g., downloadable app) may be stored at least semi-permanently or may be temporarily generated in a storage medium, such as a memory of a server of a manufacturer, a server of an application store, or a relay server.

The above-described embodiments are merely specific examples to describe technical content according to the embodiments of the disclosure and help the understanding of the embodiments of the disclosure, not intended to limit the scope of the embodiments of the disclosure. Accordingly, the scope of various embodiments of the disclosure should be interpreted as encompassing all modifications or variations derived based on the technical spirit of various embodiments of the disclosure in addition to the embodiments disclosed herein.

What is claimed is:

1. A sleep management system, comprising:
a plurality of sensors comprising a first sensor and a second sensor, the first sensor and the second sensor being configured to collect data of a user;
a hub device configured to:
receive first data from the first sensor and second data from the second sensor,
obtain first processed data based on processing of the first data, and
obtain second processed data based on processing of the second data; and
a user device configured to:
receive the first processed data and the second processed data from the hub device,
obtain sleep state information about a sleep stage and a body movement of the user based on the first processed data and the second processed data,
determine whether a Rapid Eye Movement Sleep Behavior Disorder (RBD) of the user occurs based on the body movement of the user and the sleep stage of the user, and
perform a first operation based on an occurrence of the RBD.

2. The sleep management system of claim 1, wherein the plurality of sensors comprise at least two among a pressure sensor, a Ultra-Wideband (UWB) sensor, a radar sensor, a photoplethysmography (PPG) sensor, an electrocardiogram sensor, and an acceleration sensor.

3. The sleep management system of claim 1, wherein the hub device is further configured to:
obtain the first processed data by inputting the first data to a first machine learning model, and
obtain the second processed data by inputting the second data to a second machine learning model.

4. The sleep management system of claim 1, wherein the user device is further configured to determine that the RBD occurs based on the sleep stage of the user being a Rapid Eye Movement (REM) sleep stage and the body movement of the user being greater than a preset movement.

5. The sleep management system of claim 1, wherein the first sensor is a contact sensor, and the second sensor is a non-contact sensor.

6. The sleep management system of claim 5, wherein the user device is further configured to:
based on the sleep stage of the user being the REM sleep stage, obtain information about a first body movement of the user by inputting the first processed data, corresponding to the first data collected by the contact sensor, to a machine learning model,
obtain information about a second body movement of the user by inputting the second processed data, corresponding to the second data collected by the non-contact sensor, to the machine learning model, and
determine whether the RBD occurs based on the first body movement and the second body movement being greater than a preset movement.

7. The sleep management system of claim 1, wherein the first operation comprises an operation of alerting the user of the occurrence of the RBD via a user interface.

8. The sleep management system of claim 1, wherein the first operation comprises an operation of notifying a medical institution of the occurrence of the RBD.

9. The sleep management system of claim 1, further comprising:
a server device configured to receive RBD information from the user device.

10. The sleep management system of claim 9, wherein the server device is further configured to control at least one home appliance to perform a second operation to alleviate the RBD based on the occurrence of the RBD.

11. The sleep management system of claim 9, wherein the server device is further configured to control at least one home appliance to perform a third operation to wake up the user based on the occurrence of the RBD.

12. The sleep management system of claim 9, wherein the user device is further configured to:
based on the occurrence of the RBD, perform at least one of an operation of alerting the user of the occurrence of RBD, or an operation of transmitting the RBD information to a medical institution.

13. A sleep management method, comprising:
receiving, by a hub device, first data from a first sensor and second data from a second sensor;
obtaining, by the hub device, first processed data based on processing of the first data, and second processed data based on processing of the second data;
transmitting, by the hub device, the first processed data and the second processed data to a user device;
obtaining, by the user device, sleep state information about a sleep stage and a body movement of a user based on the first processed data and the second processed data received from the hub device;
determining, by the user device, whether a Rapid Eye Movement Sleep Behavior Disorder (RBD) of the user occurs based on the body movement of the user and the sleep stage of the user; and
performing, by the user device, a first operation based on an occurrence of the RBD.

14. The sleep management method of claim 13, wherein the determining whether the RBD of the user occurs comprises:
determining that the sleep stage of the user is the Rapid Eye Movement (REM) sleep stage and the body movement of the user is greater than a preset movement.

15. The sleep management method of claim 13, wherein the first sensor is a contact sensor, the second sensor is a non-contact sensor, and
wherein the determining whether the RBD of the user occurs comprises:
based on the sleep stage of the user being the REM sleep stage, obtaining information about a first body movement of the user by inputting the first processed data, corresponding to the first data collected by the contact sensor, to a machine learning model,
obtaining information about a second body movement of the user by inputting the second processed data, corresponding to the second data collected by the non-contact sensor, to the machine learning model, and
determining that the RBD occurs based on the first body movement and the second body movement being greater than a preset movement.

16. A non-transitory computer-readable medium having instructions stored therein, which when executed by one or more processors cause the one or more processors to execute a sleep management method, the sleep management method comprising:
receiving, by a hub device, first data from a first sensor and second data from a second sensor;
obtaining, by the hub device, first processed data based on processing of the first data, and second processed data based on processing of the second data;
transmitting, by the hub device, the first processed data and the second processed data to a user device;
obtaining, by the user device, sleep state information about a sleep stage and a body movement of a user based on the first processed data and the second processed data received from the hub device;
determining, by the user device, whether a Rapid Eye Movement Sleep Behavior Disorder (RBD) of the user occurs based the sleep state information; and
performing at least one operation based on an occurrence of the RBD.

17. The non-transitory computer-readable medium of claim 16,
wherein the determining whether a RBD of the user occurs comprises:
determining that the sleep stage of the user is the Rapid Eye Movement (REM) sleep stage and the body movement of the user is greater than a preset movement.

18. The non-transitory computer-readable medium of claim 16,
wherein the at least one operation comprises one or more of:
notifying a medical institution of the occurrence of the RBD,
notifying the user of the occurrence of RBD, and
control at least one home appliance to address the occurrence of the RBD.
